(19) United States
(12) Patent Application Publication
(10) Pub. No.: US 2014/0371123 A1
(43) Pub. Date: Dec. 18, 2014

(54) AQUEOUS OPHTHALMIC COMPOSITION

(71) Applicant: ROHTO PHARMACEUTICAL CO., LTD., Osaka (JP)

(72) Inventor: Atsuko NAKATA, Osaka (JP)

(21) Appl. No.: 14/477,502

(22) Filed: Sep. 4, 2014

Related U.S. Application Data
(62) Division of application No. 13/720,799, filed on Dec. 19, 2012.
(60) Provisional application No. 61/667,665, filed on Jul. 3, 2012.

(30) Foreign Application Priority Data
Jun. 27, 2012 (JP) 2012-144223

(51) Int. Cl. A61L 12/14

(52) U.S. Cl. CPC A61L 12/14
USPC 510/112

(57) ABSTRACT

This invention relates to an aqueous ophthalmic composition comprising (A) polyoxyethylene castor oil in which the number of moles of added ethylene oxide is 2 to 30, and (B) at least one member selected from the group consisting of castor oil and vitamin A. According to the aqueous ophthalmic composition of the present invention, the defoaming time can be reduced even when foam is generated by vibration or impact.

### BACKGROUND OF INVENTION

1. Technical Field

The present invention relates to an aqueous ophthalmic composition. More specifically, the present invention relates to an aqueous ophthalmic composition having a reduced defoaming time.

2. Background Art

In the field of ophthalmology, solubilizing agents are added to a variety of preparations. In particular, in aqueous ophthalmic compositions, various solubilizing agents are added for the purpose of helping dissolution of biologically active components and additives with relatively low water solubility, and the like. A surfactant can be given as an example of the solubilizing agents used in the field of ophthalmology. It is known that polyoxyethylene castor oil is a nonionic surfactant and is added to an aqueous ophthalmic composition to help dissolve other components (JP2005-298448A).

An aqueous composition containing a surfactant is known to easily foam, and foam is generated when vibration or impact is applied during production or distribution. In general, to use the aqueous ophthalmic composition in a manner safe on the eyes, the dissolution check during production is considered important. Of aqueous ophthalmic compositions, medical products such as eye drops and eye washes require foreign matter detection in the production steps. However, when foam is generated in the aqueous ophthalmic composition during production, and disappears at low speed, it is hard to distinguish active ingredients or foreign matter from the foam. Consequently, steps such as dissolution check and foreign matter detection take a long period of time, which prevents efficient production.

On the other hand, to improve the properties of aqueous ophthalmic compositions, addition of various components has been attempted. For example, as a method for stabilizing the viscosity of a composition, JP2006-117656A discloses a castor oil-containing composition that is applicable to mucous membranes. Further, vitamin A has been added to ophthalmologic compositions in order to promote metabolism, cellular respiration, etc., of eye cells to relieve eyestrain or exhibit anti-inflammatory action (JP2009-173638A).

However, an effect on the defoaming time attained by adding such components to an aqueous ophthalmic composition has not yet been clarified. In particular, an effect on an aqueous ophthalmic composition provided when these components and a specific surfactant are added to the aqueous ophthalmic composition cannot be easily predicted.

### SUMMARY OF INVENTION

Because of the importance of steps such as dissolution check and foreign matter detection in the aqueous ophthalmic composition described above, reducing the defoaming time is an important object in the aqueous ophthalmic composition. The present invention was made in light of such prior art and provides an aqueous ophthalmic composition having a reduced defoaming time when foam is generated by vibration or impact, wherein the aqueous ophthalmic easily generates foam due to inclusion of a solubilizing agent such as a surfactant. The present invention also provides a method for reducing the defoaming time in the aqueous ophthalmic composition.

To achieve the above objects, the present inventors conducted extensive research. As a result, they found the following. When foam is generated in an aqueous ophthalmic composition by vibration or impact, the defoaming time can be significantly reduced by adding at least one member (hereinafter sometimes referred to as “component (B)”) selected from the group consisting of castor oil and vitamin A to an aqueous ophthalmic composition containing, as a nonionic surfactant, polyoxyethylene castor oil (hereinafter sometimes simply referred to as “component (A)”) in which the number of moles of added ethylene oxide is 2 to 30. This makes it possible to perform dissolution check and foreign matter detection in a short period of time. Further, an aqueous ophthalmic composition such as eye drops in which foam is generated shows large variation in the drip amount per use. In particular, for eye drops or solutions for wearing a contact lens used in a relatively small amount each time, users have difficulty controlling the amount used per use, thus causing disadvantages such as difficulty in handling. In particular, when the aqueous ophthalmic composition is used as a medical product, compliance may be reduced. According to the present invention, since the defoaming time is reduced, variation in the drip amount of the aqueous ophthalmic composition can also be reduced.

The present invention was accomplished as a result of further research based on these findings.

Accordingly, the present invention provides aqueous ophthalmic compositions according to the following embodiments.

Item 1-1. An aqueous ophthalmic composition comprising (A) polyoxyethylene castor oil in which the number of moles of added ethylene oxide is 2 to 30, and (B) at least one member selected from the group consisting of castor oil and vitamin A.
Item 1-2. The aqueous ophthalmic composition according to Item 1-1, wherein component (A) is polyoxyethylene castor oil in which the average number of moles of added ethylene oxide is 2 to 12.
Item 1-3. The aqueous ophthalmic composition according to Item 1-1 or Item 1-2, wherein the total content of component (A) is 0.0005 to 5 w/v % based on the total amount of the aqueous ophthalmic composition.
Item 1-4. The aqueous ophthalmic composition according to any one of Items 1-1 to 1-3, wherein the total content of component (B) is 0.0001 to 5 w/v % based on the total amount of the aqueous ophthalmic composition.
Item 1-5. The aqueous ophthalmic composition according to any one of Items 1-1 to 1-4, wherein the total content of vitamin A is 200 to 5,000,000 IU/100 mL based on the total amount of the aqueous ophthalmic composition.
Item 1-6. The aqueous ophthalmic composition according to any one of Items 1-1 to 1-5, wherein the total content of component (B) is 0.00002 to 10,000 parts by weight relative to 1 part by weight of the total content of component (A).
Item 1-7. The aqueous ophthalmic composition according to any one of Items 1-1 to 1-6, which further comprises at least one member (hereinbelow sometimes referred to as “component (C)”) selected from the group consisting of boric acids and salts thereof.
Item 1-8. The aqueous ophthalmic composition according to Item 1-7, wherein the total content of component (C) is 0.01 to 10 w/v % based on the total amount of the aqueous ophthalmic composition.
Item 1-9. The aqueous ophthalmic composition according to any one of Items 1-1 to 1-8, which further comprises a buffer.
Item 1-10. The aqueous ophthalmic composition according to any one of Items 1-1 to 1-9, which further comprises a nonionic surfactant other than component (A).
Item 1-11. The aqueous ophthalmic composition according to Item 1-10, wherein the nonionic surfactant other than component (A) is at least one member selected from the group consisting of polyoxyethylene sorbitan fatty acid esters, polyoxyethylene hydrogenated castor oils, polyoxyethylene castor oils in which the number of moles of added ethylene oxide exceeds 30, and polyoxyethylene-polyoxypropylene block copolymers.
Item 1-12. The aqueous ophthalmic composition according to Item 1-10 or 1-11, wherein the total content of the nonionic surfactant other than component (A) is 0.001 to 3 w/v % based on the total amount of the aqueous ophthalmic composition.
Item 1-13. The aqueous ophthalmic composition according to any one of Items 1-1 to 1-12, which further comprises a tonicity agent.
Item 1-14. The aqueous ophthalmic composition according to Item 1-13, wherein the tonicity agent is at least one member selected from the group consisting of glycerin, propylene glycol, glucose, sodium chloride, potassium chloride, calcium chloride, and magnesium chloride.
Item 1-15. The aqueous ophthalmic composition according to Item 1-13 or 1-14, wherein the total content of the tonicity agent is 0.005 to 10 w/v % based on the total amount of the aqueous ophthalmic composition.
Item 1-16. The aqueous ophthalmic composition according to any one of Items 1-1 to 1-9, which further comprises at least one member (hereinbelow sometimes referred to as “component (D)”) selected from the group consisting of glycerin and nonionic surfactants other than component (A).
Item 1-17. The aqueous ophthalmic composition according to Item 1-16, wherein component (D) is at least one member selected from the group consisting of glycerin, polyoxyethylene sorbitan fatty acid esters, polyoxyethylene hydrogenated castor oils, polyoxyethylene hydrogenated castor oils, polyoxyethylene castor oils in which the number of moles of added ethylene oxide exceeds 30, and polyoxyethylene-polyoxypropylene block copolymers.
Item 1-18. The aqueous ophthalmic composition according to Item 1-16 or 1-17, wherein the total content of component (D) is 0.01 to 10 w/v % based on the total amount of the aqueous ophthalmic composition.
Item 1-19. The aqueous ophthalmic composition according to any one of Item 1-16 to 1-18, wherein the total content of component (A) and component (D) is 2 to 50,000 parts by weight relative to 1 part by weight of the total content of component (B).
Item 1-20. The aqueous ophthalmic composition according to any one of Items 1-1 to 1-19, which is placed in a polyethylene terephthalate container.
Item 1-21. The aqueous ophthalmic composition according to any one of Items 1-1 to 1-20, which is placed in a container on which a polyethylene nozzle is mounted.
Item 1-22. The aqueous ophthalmic composition according to any one of Items 1-1 to 1-21, which is eye drops.
Item 1-23. The aqueous ophthalmic composition according to any one of Items 1-1 to 1-21, which is an eye wash.
Item 1-24. The aqueous ophthalmic composition according to any one of Items 1-1 to 1-21, which is a solution for wearing a contact lens.
Item 1-25. The aqueous ophthalmic composition according to any one of Items 1-1 to 1-21, which is a contact lens care solution.

The present invention also provides methods for reducing the defoaming time in the aqueous ophthalmic composition and a method for reducing variation in the drip amount during use according to the following embodiments.

Item 2. A method for reducing defoaming time in an aqueous ophthalmic composition, comprising adding (A) polyoxyethylene castor oil in which the number of moles of added ethylene oxide is 2 to 30, and (B) at least one member selected from the group consisting of castor oil and vitamin A to the aqueous ophthalmic composition.
Item 3. A method for reducing defoaming time in an aqueous ophthalmic composition comprising (A) polyoxyethylene castor oil in which the number of moles of added ethylene oxide is 2 to 30, comprising adding (B) at least one member selected from the group consisting of castor oil and vitamin A to the aqueous ophthalmic composition.
Item 4. A method for reducing variation in drip amount during use of an aqueous ophthalmic composition, comprising adding (A) polyoxyethylene castor oil in which the number of moles of added ethylene oxide is 2 to 30 and (B) at least one member selected from the group consisting of castor oil and vitamin A to the aqueous ophthalmic composition.

The present invention provides a method for improving transparency of the aqueous ophthalmic composition according to the following embodiment.

Item 5. A method for improving transparency of an aqueous ophthalmic composition, comprising adding (A) polyoxyethylene castor oil in which the number of moles of added ethylene oxide is 2 to 30, (B) at least one member selected from the group consisting of castor oil and vitamin A, and (D) at least one member selected from the group consisting of glycerin and nonionic surfactants other than component (A) to the aqueous ophthalmic composition.

Further, the present invention provides use according to the following embodiments.

Item 6. Use of (A) polyoxyethylene castor oil in which the number of moles of added ethylene oxide is 2 to 30 and (B) at least one member selected from the group consisting of castor oil and vitamin A, for production of an aqueous ophthalmic composition.
Item 7. The use according to Item 6, wherein the aqueous ophthalmic composition is the composition according to any one of Items 1-1 to 1-25.

The present invention also provides use according to the following embodiments.

Item 8. Use of a composition as an aqueous ophthalmic composition, the composition comprising (A) polyoxyethylene castor oil in which the number of moles of added ethylene oxide is 2 to 30 and (B) at least one member selected from the group consisting of castor oil and vitamin A.
Item 9. The use according to Item 8, wherein the composition is the composition according to any one of Items 1-1 to 1-25.

Furthermore, the present invention provides compositions according to the following embodiments.

Item 10. A composition for use as an aqueous ophthalmic composition, the composition comprising (A) polyoxyethylene castor oil in which the number of moles of added ethylene oxide is 2 to 30 and (B) at least one member selected from the group consisting of castor oil and vitamin A.
Item 11. The composition according to Item 10, which is recited in any one of Items 1-1 to 1-25.

The present invention provides methods for producing an aqueous ophthalmic composition according to the following embodiments.

Item 12. A method for producing an aqueous ophthalmic composition, comprising adding (A) polyoxyethylene castor oil in which the number of moles of added ethylene oxide is 2 to 30 and (B) at least one member selected from the group consisting of castor oil and vitamin A to a carrier containing water.
Item 13. The method according to Item 12, wherein the aqueous ophthalmic composition is the composition according to any one of Items 1-1 to 1-25.

### Advantageous Effects of Invention

According to the present invention, the defoaming time in the aqueous ophthalmic composition containing polyoxyethylene castor oil in which the average number of moles of added ethylene oxide is 2 to 30 can be reduced. As a result, dissolution check or foreign matter detection during the production of the aqueous ophthalmic composition can be performed in a short period of time, thereby allowing the production efficiency to be improved. Further, variation in the amount of drip can be reduced by shortening the defoaming time.

Further, the transparency of the aqueous ophthalmic composition of the present invention is improved and turbidity is reduced by adding at least one member selected from the group consisting of glycerin and nonionic surfactants other than polyoxyethylene castor oil, in addition to polyoxyethylene castor oil in which the average number of moles of added ethylene oxide is 2 to 30 and at least one member selected from the group consisting of castor oil and vitamin A. Therefore, the aqueous ophthalmic composition has a particularly preferable quality and appearance as an aqueous ophthalmic composition. As a result, dissolution check or foreign matter detection during the production of the aqueous ophthalmic composition can be performed in a short period of time, which enables improvement in the production efficiency.

### DESCRIPTION OF EMBODIMENTS

In the present specification, the unit of content “%” indicates w/v % and is same as g/100 mL.

In the present specification, the abbreviation “POE” means polyoxyethylene unless otherwise specified.

In the present specification, the abbreviation “POP” means polyoxypropylene unless otherwise specified.

In the present specification, contact lenses include various types of contact lenses such as hard lenses, oxygen-permeable hard lenses, soft lenses (including silicone hydrogel lenses), and color lenses unless otherwise specified.

The present invention is explained in detail below.

### 1. Aqueous Ophthalmic Composition

The aqueous ophthalmic composition of the present invention contains polyoxyethylene castor oil (component (A)) in which the average number of moles of added ethylene oxide is 2 to 30. By using the polyoxyethylene castor oil in combination with at least one member selected from the group consisting of castor oil and vitamin A described below, the present invention can attain the aforementioned excellent effects.

Polyoxyethylene castor oil is a known compound obtained by addition polymerization of ethylene oxide with castor oil, and several kinds of polyoxyethylene castor oils having a different average number of moles of added ethylene oxide are known. In the present invention, polyoxyethylene castor oil in which the average number of moles of added ethylene oxide is 2 to 30 is used as component (A). Specifically, it is possible to use polyoxyethylene castor oil 3 in which the average number of moles of added ethylene oxide is 3, polyoxyethylene castor oil 4 in which the average number of moles of added ethylene oxide is 4, polyoxyethylene castor oil 6 in which the average number of moles of added ethylene oxide is 6, polyoxyethylene castor oil 7 in which the average number of moles of added ethylene oxide is 7, polyoxyethylene castor oil 10 in which the average number of moles of added ethylene oxide is 10, polyoxyethylene castor oil 13.5 in which the average number of moles of added ethylene oxide is 13.5, polyoxyethylene castor oil 17 in which the average number of moles of added ethylene oxide is 17, polyoxyethylene castor oil 20 in which the average number of moles of added ethylene oxide is 20, polyoxyethylene castor oil 25 in which the average number of moles of added ethylene oxide is 25, and polyoxyethylene castor oil 30 in which the average number of moles of added ethylene oxide is 30.

Of these polyoxyethylene castor oils, polyoxyethylene castor oil in which the average number of moles of added ethylene oxide is 2 to 20, and preferably 2 to 12, is an example of the polyoxyethylene castor oils that exhibit particularly excellent effects of the invention.

In the present invention, these polyoxyethylene castor oils may be used singly or in any combination of two or more. Note that polyoxyethylene castor oil used in the present invention is a compound that is different from and can be distinguished from polyoxyethylene hydrogenated castor oil obtained by addition polymerization of hydrogenated castor oil with ethylene oxide.

The content of component (A) in the aqueous ophthalmic composition of the present invention is not particularly limited and is suitably determined according to the kind of component (A), kind and content of component (B) used in combination with component (A), application, preparation form, usage, etc. of the aqueous ophthalmic composition. For example, the total content of component (A) is 0.0005 to 5 w/v %, preferably 0.001 to 4 w/v %, more preferably 0.002 to 3 w/v %, even more preferably 0.005 to 2, and particularly preferably 0.01 to 0.8 w/v % based on the total amount of the aqueous ophthalmic composition of the present invention.

The aforementioned content of component (A) is preferable to further improve the effect of reducing the defoaming time in the aqueous ophthalmic composition.

It is necessary for the aqueous ophthalmic composition of the present invention to include, in addition to component (A), at least one component (component (B)) selected from the group consisting of castor oil and vitamin A. Thus, by the combined use of component (A) and component (B), the effect of reducing the defoaming time can be exhibited.

Castor oil means a vegetable oil obtained from seeds of plants belonging to the genus ricinus of the family Euphorbiaceae, e.g., Ricinus communis Linn (Euphorbiaceae).

Castor oil used in the aqueous ophthalmic composition of the present invention is not particularly limited as long as it is a pharmacologically (pharmaceutically) or physiologically acceptable oil in the field of medicine. Castor oil obtained from seeds by using a known exploitation method or known purification method, or commercially available oil can be used.

Vitamin A is a lipid-soluble vitamin and an indispensable nutrient for the human body. As vitamin A, both natural and synthetic compounds having vitamin A activity can be used.

Vitamin A used in the present invention is not particularly limited as long as it is pharmacologically (pharmaceutically) or physiologically acceptable in the field of medicine. Specific examples thereof include retinol, retinal, retinoic acid, derivatives and salts thereof, and the like. Examples of the vitamin A in a derivative form include retinol pulmitate, retinol acetate, retinol butyrate, retinol propionate, retinol octylate, retinol laurate, retinol oleate, retinol linolenate, retinal, retinoic acid, methyl retinoate, ethyl retinoate, retinol retinoate, δ-tocopheryl retinoate, α-tocopheryl retinoate, β-tocopheryl retinoate, and the like.

Vitamin A in the form of a salt is not particularly limited as long as it is pharmacologically (pharmaceutically) or physiologically acceptable in the field of medicine.

Specific examples thereof include organic acid salts such as monocarboxylic acid salts (e.g., acetic acid salt, trifluoroacetic acid salt, butyric acid salt, pulmitic acid salt, and stearic acid salt), multivalent carboxylic acid salts (e.g., fumaric acid salt, maleic acid salt, succinic acid salt, and malonic acid salt), oxycarboxylic acid salts (e.g., lactic acid salt, tartaric acid salt, and citric salt), and organic sulfonic acid salts (e.g., methanesulfonic acid salt, toluenesulfonic acid salt, and tosic acid salt); inorganic acid salts (e.g., hydrochloric acid salt, sulfuric acid salt, nitric acid salt, hydrobromic acid salt, and phosphoric acid salt), salts with an organic base (e.g., salts with organic amine such as methylamine, triethylamine, triethanol amine, morpholine, piperazine, pyrrolidine, tripyridine, and picoline); salts with an inorganic base (e.g., ammonium salt; a salt with a metal such as an alkali metal (sodium, potassium, etc.), alkaline earth metal (calcium, magnesium, etc.) or aluminum.)

Vitamin A may be isolated from natural materials such as animal sources, or synthesized by a chemical method. Vitamin A can also be used as vitamin A oil in which vitamin A is dissolved in oil. Vitamin A oil may be natural oil that is extracted from animals and then purified, or it may be oil in which vitamin A is dissolved in vegetable oil.

Such vitamin As may be used singly or in a combination of two or more. Of the vitamin As, retinol acetate, retinol pulmitate, and vitamin A oil are preferable to further improve the effect of reducing the defoaming time.

The content of component (B) in the aqueous ophthalmic composition is not particularly limited and can be suitably determined according to the kind of component (B), kind and content of component (A) used in combination with component (B), and the application, preparation form, usage, etc. of the aqueous ophthalmic composition. For example, the total content of component (B) is 0.0001 to 5 w/v %, preferably 0.0002 to 1 w/v %, more preferably 0.0005 to 0.3 w/v %, and particularly preferably 0.001 to 0.1 w/v % based on the total amount of the aqueous ophthalmic composition of the present invention.

As those skilled in the art generally understand, IU is known as an international unit for the amount of vitamin A. For example, it is well known that in the case of retinol, one IU is equivalent to about 0.30 μg of retinol, in the case of retinol acetate, one IU is equivalent to about 0.34 μg of retinol acetate, and in the case of retinol palmitate, one IU is equivalent to about 0.55 μg of retinol palmitate.

When the content of vitamin A is represented by IU, for example, the total content of vitamin A is 200 to 5,000,000 IU/100 mL, preferably 1,000 to 1,000,000 IU/100 mL, more preferably 5,000 to 500,000 IU/100 mL, and particularly preferably 10,000 to 100,000 IU/100 mL, based on the total amount of the aqueous ophthalmic composition of the present invention.

The content of component (B) is preferable to further improve the effect of the present invention.

The content ratio of component (B) to component (A) in the aqueous ophthalmic composition of the present invention is not particularly limited and is suitably determined according to the kinds of components (A) and (B), and the application, preparation form, usage, etc., of the aqueous ophthalmic composition. For example, the total content of component (B) is 0.00002 to 10,000 parts by weight, preferably 0.0001 to 1,000 parts by weight, more preferably 0.0002 to 200 parts by weight, even more preferably 0.0005 to 50 parts by weight, and particularly preferably 0.001 to 10 parts by weight relative to 1 part by weight of the total content of component (A) contained in the aqueous ophthalmic composition of the present invention.

The aforementioned content ratio of component (B) to component (A) is preferable in view of further improvement of the effect of the present invention.

As described below, various pharmacologically active components, biologically active components, etc., can be added, according to the purpose of use, to the aqueous ophthalmic composition of the present invention, and various kinds of additives can also be added. In this case, to improve the solubility of biologically active components, additives, etc., a surfactant other than component (A) can be added as a solubilizing agent. In general, the addition of such a surfactant increases foaming; however, according to the present invention, the defoaming time in an aqueous ophthalmic composition that is likely to make foam due to the addition of a surfactant other than component (A) can also be reduced by concurrently adding component (A) and component (B). The production efficiency can also be improved, and further, variation in the drip amount can be reduced.

The surfactant other than component (A), which can be added to the aqueous ophthalmic composition of the present invention, is not particularly limited as long as it is a pharmacologically (pharmaceutically) or physiologically acceptable surfactant in the field of medicine. The surfactant may be a nonionic surfactant, ampholytic surfactant, anionic surfactant, or cationic surfactant.

Specific examples of the nonionic surfactant other than component (A) that can be added to the aqueous ophthalmic composition of the present invention include POE (20) sorbitan monolaurate (polysorbate 20), POE (20) sorbitan monopalmitate (polysorbate 40), POE (20) sorbitan monostearate (polysorbate 60), POE (20) sorbitan tristearate (polysorbate 65), and POE (20) sorbitan monooleate (polysorbate 80), and like POE sorbitan fatty acid esters; POE (40) hydrogenated castor oil (polyoxyethylene hydrogenated castor oil 40), POE (60) hydrogenated castor oil (polyoxyethylene hydrogenated castor oil 60), and like POE hydrogenated castor oils; POE (9) lauryl ether, and like POE alkyl ethers; POE (20) POP (4) cetyl ether, and like POE-POP alkyl ethers; POE (196) POP (67) glycol (poloxamer 407, pluronic F127), POE (200) POP (70) glycol, and like polyoxyethylene-polyoxypropylene block copolymers; polyoxyethylene castor oil 35, polyoxyethylene castor oil 40, polyoxyethylene castor oil 50, polyoxyethylene castor oil 60, and like polyoxyethylene castor oils in which the average number of moles of added ethylene oxide is more than 30. In the compounds listed above, each of the numbers in the parentheses show the number of moles of the added compound.

Specific examples of the ampholytic surfactant that can be added to the aqueous ophthalmic composition of the present invention include alkyldiaminoethylglycine or salts thereof (e.g. hydrochloride).

Specific examples of the cationic surfactant that can be added to the aqueous ophthalmic composition of the present invention include benzalkonium chloride, benzethonium chloride, and the like.

Specific examples of the anionic surfactant that can be added to the aqueous ophthalmic composition of the present invention include alkylbenzene sulfonate, alkyl sulfate, polyoxyethylene alkyl sulfate, aliphatic α-sulfomethyl ester, α-olefin sulfonic acid, and the like.

A preferable surfactant other than component (A), which can be added to the aqueous ophthalmic composition of the present invention is a nonionic surfactant other than component (A), more preferably a POE sorbitan fatty acid ester, POE hydrogenated castor oil, and POE-POP block copolymer, and particularly preferably polysorbate 80, polyoxyethylene hydrogenated castor oil 60, and poloxamer 407.

In the aqueous ophthalmic composition of the present invention, the surfactants other than component (A) can be used singly or in a combination of two or more.

When a surfactant other than component (A) is added to the aqueous ophthalmic composition of the present invention, the content thereof is suitably determined according to the kind of the surfactant, kinds and contents of other components, and the application, preparation form, usage, etc. of the aqueous ophthalmic composition. For example, the total content of the surfactant other than component (A) is 0.001 to 3 w/v %, preferably 0.005 to 2 w/v %, more preferably 0.01 to 1 w/v %, and particularly preferably 0.05 to 1 w/v %, based on the total amount of the aqueous ophthalmic composition of the present invention.

The aqueous ophthalmic composition of the present invention preferably includes at least one member (component (C)) selected from the group consisting of boric acids and salts thereof. By including at least one member selected from the group consisting of boric acids and salts thereof, the effect of the present invention can be further improved.

Boric acid collectively refers to oxoacid generated by hydration of diboron trioxide, and examples thereof include orthoboric acid, metaboric acid, tetra boric acid, and the like. Boric acid is a known compound and may be synthesized by a known method or can be obtained as a commercially available product.

Examples of salts of boric acid include boric acid salts such as alkali metal salt of boric acid and alkaline earth metal salt of boric acid. As a boric acid salt, a hydrate of boric acid salt can also be used. Specific examples of component (C) include boric acid (orthoboric acid), sodium borate, potassium tetraborate, potassium metaborate, ammonium borate, borax, and the like. The boric acids and salts thereof can be used singly or in a combination of two or more. Preferable examples of component (C) include a combination of boric acid and a salt thereof, more preferably a combination of boric acid and an alkali metal salt of boric acid and/or an alkaline earth metal salt of boric acid, even more preferably a combination of boric acid and an alkali metal salt of boric acid, and particularly preferably a combination of boric acid and borax.

In the aqueous ophthalmic composition of the present invention, the content of component (C) is not particularly limited and can be suitably determined according to the kind of component (C), kinds and contents of other components, and the application, preparation form, usage, etc. of the aqueous ophthalmic composition. For example, based on the total amount of the aqueous ophthalmic composition of the present invention, the total content of component (C) is 0.01 to 10 w/v %, preferably 0.05 to 5 w/v %, more preferably 0.1 to 3 w/v %, and particularly preferably 0.2 to 2 w/v %.

The aqueous ophthalmic composition of the present invention can further include a buffer. Thereby, the pH of the aqueous ophthalmic composition of the present invention can be adjusted. A buffer that can be added to the aqueous ophthalmic composition of the present invention is not particularly limited as long as it is a pharmacologically (pharmaceutically) or physiologically acceptable buffer in the field of medicine. Examples of such a buffer include phosphoric acid buffers, carbonic acid buffers, citric acid buffers, acetic acid buffers, tris buffers, aspartic acids, aspartic acid salts, epsilon-aminocaproic acids, and the like. These buffers can be used singly or in a combination of two or more. Examples of phosphoric acid buffers include phosphoric acid or phosphoric acid salts such as alkali metal phosphate and alkali earth metal phosphate. Examples of carbonic acid buffers include carbonic acid or carbonic acid salts such as alkali metal carbonate and alkali earth metal carbonate. Examples of citric acid buffers include citric acid or alkali metal citrate, alkaline earth metal citrate, and the like. As the phosphoric acid buffer, hydrate of phosphoric acid salt can also be used. More specifically, examples of the phosphoric acid buffer include phosphoric acid or salts thereof (disodium hydrogen phosphate, sodium dihydrogen phosphate, potassium dihydrogen phosphate, trisodium phosphate, dipotassium phosphate, calcium monohydrogen phosphate, calcium dihydrogenphosphate, etc.); examples of the carbonic acid buffer include carbonic acid or salts thereof (sodium bicarbonate, sodium carbonate, ammonium carbonate, potassium carbonate, calcium carbonate, potassium bicarbonate, magnesium carbonate, etc.); examples of the citric acid buffer include citric acid or salts thereof (sodium citrate, potassium citrate, calcium citrate, sodium dihydrogen citrate, disodium citrate, etc.); examples of the acetic acid buffer include acetic acid or salts thereof (ammonium acetate, potassium acetate, calcium acetate, sodium acetate, etc.); aspartic acid or salts thereof (sodium aspartate, magnesium aspartate, potassium aspartate, etc.), epsilon aminocaproic acid, and the like. Of these buffers, phosphoric acid buffers (combination of disodium hydrogen phosphate and sodium dihydrogenphosphate) are preferable.

The aqueous ophthalmic composition of the present invention may further include a tonicity agent. The tonicity agent that can be added to the aqueous ophthalmic composition of the present invention is not particularly limited as long as it is a pharmacologically (pharmaceutically) or physiologically acceptable tonicity agent in the field of medicine. Examples of the tonicity agent include disodium hydrogen phosphate, sodium dihydrogen phosphate, potassium dihydrogen phosphate, sodium hydrogen sulfite, sodium sulfite, potassium chloride, calcium chloride, sodium chloride, magnesium chloride, potassium acetate, sodium acetate, sodium bicarbonate, sodium carbonate, sodium tiosulfate, magnesium sulfate, glycerin, propylene glycol, polyethylene glycol, glucose, mannitol, sorbitol, and the like. Of these tonicity agents, preferable examples include glycerin, propylene glycol, glucose, sodium chloride, potassium chloride, calcium chloride, and magnesium chloride. These tonicity agents can be used singly or in a combination of two or more.

When a tonicity agent is added to the aqueous ophthalmic composition of the present invention, the content thereof is suitably determined according to the kind of the tonicity agent, kinds and contents of other components, and the application, preparation form, usage, etc. of the aqueous ophthalmic composition. For example, the total content of the tonicity agent is 0.005 to 10 w/v %, preferably 0.01 to 5 w/v %, and more preferably 0.05 to 3 w/v % based on the total amount of the aqueous ophthalmic composition of the present invention.

When the aqueous ophthalmic composition of the present invention contains at least one member (component D) selected from the group consisting of glycerin and nonionic surfactants other than component (A) in addition to component (A) and component (B), transparency is significantly improved, and turbidity is reduced. Thus, an aqueous ophthalmic composition having preferable quality and appearance can be obtained. Consequently, dissolution check or foreign matter detection during the production of the aqueous ophthalmic composition can be performed for a short period of time, and the production efficiency can be improved.

Preferable examples of compound (D) that is effective for improving the transparency of the aqueous ophthalmic composition of the present invention include glycerin, POE sorbitan fatty acid esters, POE hydrogenated castor oils, and POE-POP block copolymers, and more preferably glycerin, polysorbate 80, polyoxyethylene hydrogenated castor oil 60, and poloxamer 407.

When component (D) is added to the aqueous ophthalmic composition of the present invention, the content thereof is not particularly limited and can be suitably determined according to the kind of component (D), the kinds and contents of other components, and the application, preparation form, usage, etc. of the aqueous ophthalmic composition. In particular, to improve the transparency of the aqueous ophthalmic composition of the present invention, for example, the total content of component (D) is 0.001 to 10 w/v %, preferably 0.005 to 7.5 w/v %, more preferably 0.01 to 5 w/v %, even more preferably 0.02 to 4 w/v %, and particularly preferably 0.05 to 3 w/v % based on the total amount of the aqueous ophthalmic composition of the present invention.

When component (D) is added to the aqueous ophthalmic composition of the present invention, the content ratio of component (D) to component (A) and component (B) is not particularly limited and can be suitably determined according to the kinds of component (A), component (B), and component (D), and the application, preparation form, usage, etc. of the aqueous ophthalmic composition. In particular, in the aqueous ophthalmic composition of the present invention, to maintain high transparency, the ratio of the total content of components (A) and (D) relative to 1 part by weight of the total content of component (B) contained in the aqueous ophthalmic composition of the present invention is preferably 2 to 50,000 parts by weight, more preferably 3 to 10,000 parts by weight, and more preferably 4 to 2,000 parts by weight.

The pH of the aqueous ophthalmic composition is not particularly limited as long as it is within a pharmacologically (pharmaceutically) or physiologically acceptable range in the field of medicine. For example, the pH of the aqueous ophthalmic composition of the present invention is in the range of 4.0 to 9.5, preferably 5.0 to 9.0, and more preferably 5.5 to 8.5.

The osmotic pressure of the aqueous ophthalmic composition of the present invention is not particularly limited as long as it is within a range acceptable to the human body. For example, the osmotic pressure ratio of the aqueous ophthalmic composition of the present invention is 0.5 to 5.0, preferably 0.6 to 3.0, more preferably 0.7 to 2.0, and particularly preferably 0.9 to 1.55. The osmotic pressure is adjusted using an inorganic salt, polyhydric alcohol, sugar alcohol, sugar, etc., according to a known method in the technical field of the present invention. The osmotic pressure ratio is the ratio of the osmotic pressure of a sample to 286 mOsm (osmotic pressure of 0.9 w/v % aqueous sodium chloride solution) based on the Japanese Pharmacopoeia, 16^{th} revision, and can be measured with reference to the osmotic measurement method (freezing point depression method) described in the Japanese Pharmacopoeia. The reference solution for measuring the ratio of osmotic pressure (0.9 w/v % aqueous sodium chloride solution) can be prepared as follows: after sodium chloride (standard reagent according to the Japanese Pharmacopoeia is dried for 40 to 50 minutes at 500 to 650° C., the sodium chloride is allowed to cool in a desiccator (silica gel), and 0.900 g of the resultant is accurately measured. The resultant is then dissolved in purified water, thus preparing 100 mL of the solution with accuracy. Alternatively, a commercially available reference solution for measuring the osmotic pressure ratio (0.9 w/v % aqueous sodium chloride solution) can be used.

The viscosity of the aqueous ophthalmic composition of the present invention is not particularly limited as long as it is within a range acceptable to the human body. For example, the viscosity at 25° C., which is measured by a rotational viscometer (RE550 type viscometer), produced by Toki Sangyo Co., Ltd., rotor: 1° 34′×24) is 0.01 to 1,000 mPa·s, preferably 0.05 to 100 mPa·s, and more preferably 0.1 to 10 mPa·s.

As long as the aqueous ophthalmic composition of the present invention attains the effect of the present invention, it may contain, in addition to the aforementioned components, a suitable amount of various pharmacologically active components and/or biologically active components singly or in a combination. Such components are not particularly limited, and examples of the specific components used in an opthalmological drug are as follows:

antihistamines or antiallergic agents such as iproheptine, diphenhydramine hydrochloride, chlorphenylamine maleate, ketotifen fumarate, pemirolast potassium, and sodium cromoglycate;

decongestants such as tetrahydrozoline hydrochloride, naphazoline hydrochloride, naphazoline sulfate, epinephrine hydrochloride, ephedrine hydrochloride, and methylephedrine hydrochloride;

vitamins such as flavin adenine dinucleotide sodium, cyanocobalamin, pyridoxine hydrochloride, pantenol, calcium pantothenate, and tocopherol acetate;

amino acids such as potassium aspartate, magnesium aspartate, epsilon-aminocaproic acid, and sodium chondroitin sulfate;

antiphlogistics such as bromfenac sodium, dipotassium glycyrrhizate, pranoprofen, allantoin, berberine chloride, berberine sulfate, lysozyme chloride, and licorice; and

others such as sodium hyaluronate, sulfamethoxazole, and sulfamethoxazole sodium.

Further, in the aqueous ophthalmic composition of the present invention, as long as the effect of the invention is attained, a suitable amount of one or more additives selected from various additives can be suitably added by a conventional method according to the application, preparation form, etc. of the aqueous ophthalmic composition. Typical components include the following additives:

carriers such as water, moisture ethanol, and like aqueous carriers:

sugars such as cyclodextrin;

sugar alcohols such as xylitol, sorbitol, and mannitol, wherein these compounds may be in the d form, l form, or dl form;

antiseptics, disinfectants, and antibacterial agents such as cetyl pyridinium, benzalkonium chloride, benzethonium chloride, polyhexanide hydrochloride, alkyldiaminoethylglycine hydrochloride, sodium benzoate, ethanol, chloro butanol, sorbic acid, potassium sorbate, sodium dehydroacetate, methyl parahydroxybenzoate, ethyl parahydroxybenzoate, propyl parahydroxybenzoate, butyl parahydroxybenzoate, oxyquinoline sulfate, phenethyl alcohol, benzyl alcohol, and Glokill (trade name, Rhodia Co., Ltd.);

thickening agents or thickeners such as powdered Acacia, sodium alginate, propylene glycol alginate, sodium chondroitin sulfate, sorbitol, dextran 70, powdered tragacanth, methylcellulose, carboxymethyl cellulose, hydroxyethyl cellulose, hydroxypropyl methylcellulose, carboxyvinyl polymer, polyvinyl alcohol, polyvinyl pyrrolidone, and macrogol 4000;

oils such as sesame oil; and the like.

The aqueous ophthalmic composition of the present invention can be prepared by adding a desired amount of each of component (A) and component (B), and if necessary, other components to a carrier so that the aqueous ophthalmic composition has a desired concentration. For example, eye drops, solutions for wearing a contact lens, eye washes, or contact lens care solutions are prepared by dissolving or suspending the aforementioned components in purified water, adjusting the pH and osmotic pressure to the predetermined levels, and subjecting these to sterilization treatment by filter sterilization, etc. Regarding the dissolution of components (A) and (B), and the dissolution of components with a high hydrophobic property, components having a solubilizing action such as surfactants may be added beforehand, then the mixture is stirred, after which purified water is added thereto, followed by dissolution or suspension.

Accordingly, from a different viewpoint, the present invention provides a method for producing an aqueous ophthalmic composition comprising adding (A) polyoxyethylene castor oil in which the average number of moles of added ethylene oxide is 2 to 30 and (B) at least one member selected from the group consisting of castor oil and vitamin A to a water-containing carrier.

The aqueous ophthalmic composition of the present invention indicates an ophthalmic composition in which the content of water exceeds 85 w/v % or more based on the total amount of the aqueous ophthalmic composition. The content of water in the aqueous ophthalmic composition is preferably 90 w/v % or more, more preferably 92 w/v % or more, even more preferably 94 w/v % or more, and particularly more preferably 96 w/v % or more. As the water used in the aqueous ophthalmic composition of the present invention, pharmacologically (pharmaceutically) or physiologically acceptable water in the field of medicine can be used. Examples of water include distilled water, water, purified water, sterile purified water, water for injection, distilled water for injection, and the like. The dosage form of the aqueous ophthalmic composition is not particularly limited as long as it can be used in the field of ophthalmology. The dosage form is preferably liquid. These definitions are based on the Japanese Pharmacopoeia, 16th revision.

Examples of the aqueous ophthalmic composition of the present invention include eye drops (also called as ophthalmic solutions or ophthalmic drugs) [note that examples of the eye drops include artificial tears and ophthalmic agents that can be instilled into eyes during use of contact lenses,] eye washes (also referred to as collyriums or eye lotions) [note that examples of eye washes include eye washes that can wash eyes during use of contact lenses,] solutions for wearing contact lenses, contact lens care products, (disinfectant solutions for contact lenses, storage solutions for contact lenses, cleansing solutions for contact lenses, cleansing and storage solutions for contact lenses, disinfectant/storage/cleansing solutions for contact lenses (multiple-purpose solutions for contact lenses), etc. The aqueous ophthalmic composition of the present invention ensures a reduced defoaming time, and low variation in the drip amount during use. Therefore, the present invention is preferably used in eye drops and solutions for wearing a contact lens that are used in a particularly small amount each time compared to other dosage forms. The present invention is particularly preferably used in eye drops.

Moreover, since the aqueous ophthalmic composition of the present invention ensures a reduced defoaming time, polyoxyethylene castor oil, which is used as a surfactant, can be uniformly dissolved in the aqueous ophthalmic composition in a short period of time and can sufficiently exhibit original properties of polyoxyethylene castor oil. Therefore, the aqueous ophthalmic composition of the present invention is preferably used as an aqueous ophthalmic composition that requires a sufficient cleansing effect compared to other dosage forms. From this point of view, the aqueous ophthalmic composition of the present invention is particularly preferably used as an eye wash.

As the container that holds the aqueous ophthalmic composition of the present invention, a container that can be generally used to hold an aqueous ophthalmic composition can be used. The container may be made of glass or plastic. When a plastic container is used to hold the aqueous ophthalmic composition of the present invention, although constituent materials of the plastic container are not particularly limited, polyethylenenaphthalate, polyarylate, polyethylene terephthalate, polypropylene, polyethylene, and polyimide can be used alone or in a mixture of two or more. The copolymers thereof can be also used. Examples of the copolymers include copolymers that contain other polyester units or imide units, in addition to any one of ethylene-2,6-naphthalate units, arylate units, ethylene terephthalate units, propylene units, ethylene units, and imide units, which is contained as a main component. In the present invention, for example, a polyethylene terephthalate container indicates a container in which polyethylene terephthalate is contained in an amount of 50 w/w % or more based on the total weight of the constituent materials of the container.

The structure, constituent materials, etc., of a container spout periphery such as a nozzle mounted on a container containing the aqueous ophthalmic composition of the present invention is not particularly limited. The structure of the container spout periphery such as a nozzle may be a generally applicable structure as a spout (e.g., nozzle) of a container for ophthalmic compositions (e.g., container for eye drops), and the nozzle may be integrally or separately formed with the container. Examples of the constituent materials of the spout periphery or spout (e.g., nozzle) include those mentioned in the constituent materials of the plastic containers.

In particular, to further improve flexibility, cost, and/or an effect of reducing variation in the drip amount, a spout that contains polyethylene or polypropylene as a constituent material is preferable. Examples of polyethylene include high-density polyethylene, low-density polyethylene, and the like; particularly, of these, a spout containing low-density polyethylene as a constituent material is preferable. As a spout, a nozzle used for a container of eye drops is preferable.

As a preferable combination of a container that holds the aqueous ophthalmic composition of the present invention and a container spout periphery, it is possible to use a combination of a polyethylene terephthalate container and a polyethylene container spout periphery, more preferably, a combination of a polyethylene terephthalate eye drop container and a polyethylene nozzle, and particularly more preferably, a combination of a polyethylene terephthalate eye drop container and a low-density polyethylene nozzle. Such a combination can significantly exhibit the effect of reducing variation in the drip amount in the present invention.

Since the aqueous ophthalmic composition of the present invention can reduce the defoaming time, reduce variation in the drip amount during use, and can be instilled into an eye in a specific amount per use, it is particularly suitably used as eye drops containing a pharmacologically active component and/or a biologically active component. Such eye drops can be used as eye drops for dry eyes, decongestant eye drops, antibacterial eye drops, anti-inflammatory eye drops, eye drops for relieving itchy eyes, eye drops for relieving eye strain, etc.

From a different viewpoint, the present invention also provides use of (A) polyoxyethylene castor oil in which the average number of moles of added ethylene oxide is 2 to 30 and (B) at least one member selected from the group consisting of castor oil and vitamin A, for the production of an aqueous ophthalmic composition.

From another different viewpoint, the present invention also provides use of a composition as an aqueous ophthalmic composition, the composition comprising (A) polyoxyethylene castor oil in which the average number of moles of added ethylene oxide is 2 to 30 and (B) at least one member selected from the group consisting of castor oil and vitamin A.

From still another different viewpoint, the present invention provides a composition for use as an aqueous ophthalmic composition, containing (A) polyoxyethylene castor oil in which the average number of moles of added ethylene oxide is 2 to 30, and (B) at least one member selected from the group consisting of castor oil and vitamin A.

### 2. Method for Reducing Defoaming Time

As mentioned above, in the aqueous ophthalmic composition of the present invention, by containing component (A) and component (B), the defoaming time can be reduced in the aqueous ophthalmic composition; consequently, variation in the drip amount during use can be reduced.

Therefore, from a different viewpoint, the present invention provides a method for reducing the defoaming time in an aqueous ophthalmic composition, comprising adding (A) polyoxyethylene castor oil in which the average number of moles of added ethylene oxide is 2 to 30, and (B) at least one member selected from the group consisting of castor oil and vitamin A, to the aqueous ophthalmic composition.

The present invention also provides a method for reducing the defoaming time of an aqueous ophthalmic composition, comprising adding (B) at least one member selected from the group consisting of castor oil and vitamin A to (A) polyoxyethylene castor oil in which the average number of moles of added ethylene oxide is 2 to 30.

The present invention provides a method for reducing variation in the drip amount during use of an aqueous ophthalmic composition, comprising adding (A) polyoxyethylene castor oil in which the average number of moles of added ethylene oxide is 2 to 30 mol, and (B) at least one member selected from the group consisting of castor oil and vitamin A to the aqueous ophthalmic composition.

In these methods, as long as component (A) and component (B) are both present, they may be added at the same time or separately, and the order thereof is not particularly limited. The kinds of component (A) and component (B) to be used, the contents (or addition contents) and the ratio thereof, the kinds and contents (addition contents) of components added other than the above, the preparation form of the aqueous ophthalmic composition, the kind and combination of the container, the embodiment method, and the like are the same as in the “1. Aqueous Ophthalmic Composition” section above.

In particular, the methods described above are suitably used when the aqueous ophthalmic composition is used as eye drops or a solution for wearing a contact lens.

In the present specification, whether the defoaming time in the aqueous ophthalmic composition is shortened or not can be determined according to the method in the Examples described below.

### 3. Method for Improving Transparency

As described above, the transparency can be significantly improved by containing at least one member (component (D)) selected from the group consisting of glycerin and nonionic surfactants other than component (A), in addition to components (A) and (B) in the aqueous ophthalmic composition of the present invention, and a preferable quality and appearance as the aqueous ophthalmic composition can be attained. Consequently, dissolution check and foreign matter detection during the production of the aqueous ophthalmic composition can be performed in a short period of time, which improves production efficiency.

Accordingly, from another viewpoint, the present invention provides a method for improving the transparency of an aqueous ophthalmic composition, comprising adding (A) polyoxyethylene castor oil in which the average number of moles of added ethylene oxide is 2 to 30, (B) at least one member selected from the group consisting of castor oil and vitamin A, and (D) at least one member selected from the group consisting of glycerin and nonionic surfactants other than component (A), to the aqueous ophthalmic composition.

In this method, as long as component (A), component (B), and component (D) are present, they may be added at the same time or separately, and the order thereof is not particularly limited. The kinds of component (A), component (B), and component (D) to be used, the contents (or addition contents) and the ratio thereof, the kinds and the contents (addition contents) of components other than the above, the preparation form of the aqueous ophthalmic composition, the kind and combination of the container, the embodiment method, and the like are the same as in the “1. Aqueous Ophthalmic Composition” section above.

In the present specification, whether the transparency of the aqueous ophthalmic composition is improved or not can be determined according to the method in the Examples described below.

### EXAMPLES

Examples and Test Examples are given below to illustrate the present invention in detail; however, the present invention is not limited to these Examples and the like.

### Test Example 1

### Defoaming Time Test (1)

Aqueous ophthalmic compositions having the formulations shown in the following Tables 1 to 3 were prepared by a standard method, and defoaming times were evaluated using these compositions. Polyoxyethylene castor oil 10 that conforms to the standard for polyoxyethylene castor oil in Japanese Pharmaceutical Excipients 2003 and in which the average number of moles of added ethylene oxide is 10 was used, and castor oil manufactured by Wako was used.

Subsequently, each of the aqueous ophthalmic compositions in an amount of 30 mL was placed in individual 50-mL glass centrifuge tubes, and the tubes were shaken 1,500 times using a Recipad Shaker SR-2w (TAITEC). Immediately after shaking, a foam part and an aqueous solution part were confirmed by visual observation, and the volume of the foam part was measured. Thereafter, the tubes were allowed to stand, the volume of the foam part was measured over time, and the time required for the foam to completely disappear was measured.

Based on the defoaming time of each control and the defoaming time of each Example, reduction rates in defoaming time due to the castor oil were calculated with the following formula. A larger reduction rate means a higher foam disappearance speed.

Reduction rate in defoaming time (%)=(defoaming time of corresponding control−defoaming time of each Example)/(defoaming time of corresponding control)×100

The corresponding controls are, specifically, Control 1 for Examples 1-1 and 1-2, Control 2 for Examples 2-1 and 2-2, Control 3 for Example 3-1, and Control 4 for Example 4-1. The results are also shown in Tables 1 to 3.

TABLE 1
Unit: w/v %

| | Control 1 | Control 2 | Control 3 | Control 4 |
|---|---|---|---|---|
| Polyoxyethylene | 0.05 | 0.1 | 0.2 | 0.5 |
| castor oil 10 | | | | |
| Glycerin | 2.5 | 2.5 | 2.5 | 2.5 |
| Boric acid | 0.5 | 0.5 | 0.5 | 0.5 |
| Borax | 0.2 | 0.2 | 0.2 | 0.2 |
| Purified water | Balance | Balance | Balance | Balance |
| pH | 7.0 | 7.0 | 7.0 | 7.0 |

TABLE 2
Unit: w/v %

| | Example | Example | Example | Example |
|---|---|---|---|---|
| | 1-1 | 2-1 | 3-1 | 4-1 |
| Polyoxyethylene | 0.05 | 0.1 | 0.2 | 0.5 |
| castor oil 10 | | | | |
| Glycerin | 2.5 | 2.5 | 2.5 | 2.5 |
| Castor oil | 0.01 | 0.01 | 0.01 | 0.01 |
| Boric acid | 0.5 | 0.5 | 0.5 | 0.5 |
| Borax | 0.2 | 0.2 | 0.2 | 0.2 |
| Purified water | Balance | Balance | Balance | Balance |
| pH | 7.0 | 7.0 | 7.0 | 7.0 |
| Reduction rate | 83 | 73 | 22 | 18 |
| in defoaming | | | | |
| time (%) | | | | |

TABLE 3
Unit: w/v %

| | Example 1-2 | Example 2-2 |
|---|---|---|
| Polyoxyethylene | 0.05 | 0.1 |
| castor oil 10 | | |
| Glycerin | 2.5 | 2.5 |
| Castor oil | 0.05 | 0.05 |
| Boric acid | 0.5 | 0.5 |
| Borax | 0.2 | 0.2 |
| Purified water | Balance | Balance |
| pH | 7.0 | 7.0 |
| Reduction rate | 54 | 30 |
| in defoaming | | |
| time (%) | | |

As shown in Tables 1 to 3, it was confirmed that the defoaming times were significantly reduced in the aqueous ophthalmic compositions containing the polyoxyethylene castor oil 10 at various concentrations and containing the castor oil (Examples 1-1 to 4-1), as compared to the aqueous ophthalmic compositions containing the polyoxyethylene castor oil 10 but not containing the castor oil (Controls 1 to 4).

### Test Example 2

### Defoaming Time Test (2)

Aqueous ophthalmic compositions having the formulations shown in the following Tables 4 to 6 were prepared by a standard method, and defoaming times were evaluated. Vitamin A oil containing 55 weight % retinol palmitate, which is vitamin A, and 45 weight % sunflower oil was used. The vitamin A oil was in an amount of 1,000,000 IU/g in terms of IU, which is an international unit for the amount of vitamin A. The same polyoxyethylene castor oil 10 as in Test Example 1 was used.

Using these aqueous ophthalmic compositions, reduction rates in defoaming time due to the vitamin A oil were calculated with the following formula in the same manner as in Test Example 1. A larger reduction rate means a larger reduction in defoaming time.

Reduction rate in defoaming time (%)=(defoaming time of corresponding control−defoaming time of each Example)/(defoaming time of corresponding control)×100

The corresponding controls are, specifically, Control 5 for Examples 5-1 and 5-2, Control 6 for Examples 6-1 and 6-2, Control 7 for Example 7-1, Control 8 for Examples 8-1 and 8-2, Control 9 for Example 9-1, and Control 10 for Examples 10-1 and 10-2. The results are also shown in Tables 4 to 6.

TABLE 4
Unit: w/v %

| | Control | Control | Control | Control | Control | Control |
|---|---|---|---|---|---|---|
| | 5 | 6 | 7 | 8 | 9 | 10 |
| Polyoxy- | 0.05 | 0.1 | 0.2 | 0.3 | 0.4 | 0.5 |
| ethylene | | | | | | |
| castor oil 10 | | | | | | |
| Glycerin | 2.5 | 2.5 | 2.5 | 2.5 | 2.5 | 2.5 |
| Boric acid | 0.5 | 0.5 | 0.5 | 0.5 | 0.5 | 0.5 |
| Borax | 0.2 | 0.2 | 0.2 | 0.2 | 0.2 | 0.2 |
| Purified water | Balance | Balance | Balance | Balance | Balance | Balance |
| pH | 7.0 | 7.0 | 7.0 | 7.0 | 7.0 | 7.0 |

TABLE 5
Unit: w/v %

| | Example | Example | Example | Example | Example | Example |
|---|---|---|---|---|---|---|
| | 5-1 | 6-1 | 7-1 | 8-1 | 9-1 | 10-1 |
| Polyoxyethylene | 0.05 | 0.1 | 0.2 | 0.3 | 0.4 | 0.5 |
| castor oil 10 | | | | | | |
| Glycerin | 2.5 | 2.5 | 2.5 | 2.5 | 2.5 | 2.5 |
| Vitamin A oil | 0.01 | 0.01 | 0.01 | 0.01 | 0.01 | 0.01 |
| (1,000,000 | (10,000 | (10,000 | (10,000 | (10,000 | (10,000 | (10,000 |
| IU/g) | IU) | IU) | IU) | IU) | IU) | IU) |
| Boric acid | 0.5 | 0.5 | 0.5 | 0.5 | 0.5 | 0.5 |
| Borax | 0.2 | 0.2 | 0.2 | 0.2 | 0.2 | 0.2 |
| Purified water | Balance | Balance | Balance | Balance | Balance | Balance |
| pH | 7.0 | 7.0 | 7.0 | 7.0 | 7.0 | 7.0 |
| Reduction rate | 98 | 76 | 91 | 45 | 40 | 27 |
| in defoaming | | | | | | |
| time (%) | | | | | | |

Note:
The figures in parentheses indicate the amount of vitamin A contained in 100 mL of each composition.

TABLE 6
Unit: w/v %

| | Example | Example | Example | Example |
|---|---|---|---|---|
| | 5-2 | 6-2 | 8-2 | 10-2 |
| Polyoxyethylene | 0.05 | 0.1 | 0.3 | 0.5 |
| castor oil 10 | | | | |
| Glycerin | 2.5 | 2.5 | 2.5 | 2.5 |
| Vitamin A oil | 0.05 | 0.05 | 0.05 | 0.05 |
| (1,000,000 | (50,000 | (50,000 | (50,000 | (50,000 |
| IU/g) | IU) | IU) | IU) | IU) |
| Boric acid | 0.5 | 0.5 | 0.5 | 0.5 |
| Borax | 0.2 | 0.2 | 0.2 | 0.2 |
| Purified water | Balance | Balance | Balance | Balance |
| pH | 7.0 | 7.0 | 7.0 | 7.0 |
| Reduction rate | 91 | 88 | 99 | 80 |
| in defoaming | | | | |
| time (%) | | | | |

Note:
The figures in parentheses indicate the amount of vitamin A contained in 100 mL of each composition.

As shown in Tables 4 to 6, it was confirmed that the defoaming times were significantly reduced in the aqueous ophthalmic compositions containing the polyoxyethylene castor oil 35 at various concentrations and containing the vitamin A oil (Examples 5-1 to 10-2), as compared to the aqueous ophthalmic compositions containing the polyoxyethylene castor oil 10 but not containing the vitamin A oil (Controls 5 to 10).

### Test Example 3

### Defoaming Time Test (3)

Aqueous ophthalmic compositions having the formulations shown in the following Tables 7 and 8 were prepared by a standard method, and defoaming times were evaluated. Polyoxyethylene castor oil 35 that conforms to the standard for polyoxyethylene castor oil in Japanese Pharmaceutical Excipients 2003 and in which the average number of moles of added ethylene oxide is 35 was used. The same castor oil as in Test Example 1 was used, and the same vitamin A oil as in Test Example 2 was used.

Using these aqueous ophthalmic compositions, reduction rates in defoaming time due to the castor oil or the vitamin A oil were calculated with the following formula in the same manner as in Test Example 1. Since the defoaming times were longer than those in Test Examples 1 and 2, the time required for the initial foam to be reduced by half was evaluated as the foam half-volume period, and reduction rates thereof were calculated with the following formula. A larger reduction rate means a larger reduction in defoaming time.

Reduction rate in foam half-volume period (%)=(foam half-volume period of corresponding control−foam half-volume period of each Example)/(foam half-volume period of corresponding control)×100

The corresponding controls are, specifically, Control 11 for Comparative Example 1, Control 12 for Comparative Example 2, and Control 13 for Comparative Examples 3 and 4. The results are also shown in Tables 7 and 8.

TABLE 7
Unit: w/v %

| | Control 11 | Control 12 | Control 13 |
|---|---|---|---|
| Polyoxyethylene | 0.05 | 0.3 | 0.5 |
| castor oil 35 | | | |
| Glycerin | 2.5 | 2.5 | 2.5 |
| Boric acid | 0.5 | 0.5 | 0.5 |
| Borax | 0.2 | 0.2 | 0.2 |
| Purified water | Balance | Balance | Balance |
| pH | 7.0 | 7.0 | 7.0 |

TABLE 8
Unit: w/v %

| | Compar- | Compar- | Compar- | Compar- |
|---|---|---|---|---|
| | ative | ative | ative | ative |
| | Example 1 | Example 2 | Example 3 | Example 4 |
| Polyoxyethylene | 0.05 | 0.3 | 0.5 | 0.5 |
| castor oil 35 | | | | |
| Glycerin | 2.5 | 2.5 | 2.5 | 2.5 |
| Castor oil | 0.01 | 0.01 | 0.01 | — |
| Vitamin A oil | — | — | — | 0.01 |
| (1,000,000 | | | | (10,000 |
| IU/g) | | | | IU) |
| Boric acid | 0.5 | 0.5 | 0.5 | 0.5 |
| Borax | 0.2 | 0.2 | 0.2 | 0.2 |
| Purified water | Balance | Balance | Balance | Balance |
| pH | 7.0 | 7.0 | 7.0 | 7.0 |
| Reduction rate | 0 | 0 | −120 | 0 |
| in foam half- | | | | |
| volume period | | | | |
| (%) | | | | |

Note:
The figure in parentheses indicates the amount of vitamin A contained in 100 mL of the composition.

As shown in Tables 7 and 8, in the aqueous ophthalmic compositions containing the polyoxyethylene castor oil 35 at various concentrations and containing the castor oil or the vitamin A oil (Comparative Examples 1 to 4), the foam half-volume periods were similar to those of the aqueous ophthalmic compositions containing the polyoxyethylene castor oil 35 but containing neither the castor oil nor the vitamin A oil (Controls 11 to 13), or were extended as compared to the controls, with no reduction effect on the defoaming time being observed.

### Test Example 4

### Transparency Test

Aqueous ophthalmic compositions having the formulations shown in the following Tables 9 to 12 were prepared by a standard method, and transparency was evaluated using these compositions. The same polyoxyethylene castor oil 10 and castor oil as in Test Example 1 were used.

Subsequently, absorbance at 600 nm for each of the prepared aqueous ophthalmic compositions was measured using a Microplate Reader SH-9000 (Corona Electric). Using the measured absorbances, transmittances were calculated based on the following formula. A higher transmittance means greater suppression of white turbidity and greater transparency of the aqueous ophthalmic composition.

Transmittance (%)=(10 to the power of A₀)/(10 to the power of A)×100

A₀ indicates the absorbance of distilled water, and A indicates the absorbance of each aqueous ophthalmic composition.

Further, the transparency was evaluated according to the following criteria, and the calculated transmittances were classified. The classification results are also shown in Tables 9 to 12.

### Evaluation Criteria for Transparency

a: Transmittance of 95% or more
b: Transmittance of 90% or more and less than 95%
c: Transmittance of 85% or more and less than 90%
d: Transmittance of 75% or more and less than 85%
e: Transmittance of less than 75%

TABLE 9
Unit: w/v %

| | Comparative | Example | Example | Example | Example |
|---|---|---|---|---|---|
| | Example 5 | 11-1 | 11-2 | 11-3 | 11-4 |
| (B) Castor oil | 0.1 | 0.1 | 0.1 | 0.1 | 0.1 |
| (A) Polyoxyethylene | — | 0.01 | 0.02 | 0.05 | 0.1 |
| castor oil 10 | | | | | |
| (D) Polyoxyethylene | 0.1 | 0.1 | 0.1 | 0.1 | 0.1 |
| hydrogenated castor | | | | | |
| oil 60 | | | | | |
| Boric acid | 0.5 | 0.5 | 0.5 | 0.5 | 0.5 |
| Borax | 0.02 | 0.02 | 0.02 | 0.02 | 0.02 |
| Purified water | Balance | Balance | Balance | Balance | Balance |
| pH | 7.0 | 7.0 | 7.0 | 7.0 | 7.0 |
| ((A) + (D))/(B) | 1.0 | 1.1 | 1.2 | 1.5 | 2.0 |
| Transparency | e | d | d | d | c |

TABLE 10
Unit: w/v %

| | Comparative | Example | Example | Example | Example |
|---|---|---|---|---|---|
| | Example 6 | 12-1 | 12-2 | 12-3 | 12-4 |
| (B) Castor oil | 0.05 | 0.05 | 0.05 | 0.05 | 0.05 |
| (A) Polyoxyethylene | 0.05 | 0.03 | 0.05 | 0.05 | 0.1 |
| castor oil 10 | | | | | |
| (D) Polyoxyethylene | — | 0.05 | 0.05 | 0.1 | 0.05 |
| hydrogenated castor | | | | | |
| oil 60 | | | | | |
| Boric acid | 0.5 | 0.5 | 0.5 | 0.5 | 0.5 |
| Borax | 0.02 | 0.02 | 0.02 | 0.02 | 0.02 |
| Purified water | Balance | Balance | Balance | Balance | Balance |
| pH | 7.0 | 7.0 | 7.0 | 7.0 | 7.0 |
| ((A) + (D))/(B) | 1.0 | 1.6 | 2.0 | 3.0 | 3.0 |
| Transparency | e | d | c | b | b |

TABLE 11
Unit: w/v %

| | Example | Example | Example | Example | Example |
|---|---|---|---|---|---|
| | 13-1 | 13-2 | 13-3 | 13-4 | 13-5 |
| (B) Castor oil | 0.05 | 0.05 | 0.05 | 0.05 | 0.05 |
| (A) Polyoxyethylene | 0.05 | 0.08 | 0.1 | 0.08 | 0.1 |
| castor oil 10 | | | | | |
| (D) Polyoxyethylene | 0.2 | 0.2 | 0.2 | 0.3 | 0.3 |
| hydrogenated castor | | | | | |
| oil 60 | | | | | |
| Boric acid | 0.5 | 0.5 | 0.5 | 0.5 | 0.5 |
| Borax | 0.01 | 0.01 | 0.01 | 0.01 | 0.01 |
| Purified water | Balance | Balance | Balance | Balance | Balance |
| pH | 7.0 | 7.0 | 7.0 | 7.0 | 7.0 |
| ((A) + (D))/(B) | 5.0 | 5.6 | 6.0 | 7.6 | 8.0 |
| Transparency | a | a | a | a | a |

TABLE 12
Unit: w/v %

| | Example | Example | Example |
|---|---|---|---|
| | 14-1 | 14-2 | 14-3 |
| (B) Castor oil | 0.05 | 0.02 | 0.01 |
| (A) Polyoxyethylene | 0.1 | 0.1 | 0.1 |
| castor oil 10 | | | |
| (D) Polyoxyethylene | 0.1 | 0.1 | 0.1 |
| hydrogenated castor | | | |
| oil 60 | | | |
| Boric acid | 0.5 | 0.5 | 0.5 |
| Borax | 0.02 | 0.02 | 0.02 |
| Purified water | Balance | Balance | Balance |
| pH | 7.0 | 7.0 | 7.0 |
| ((A) + (D))/(B) | 4.0 | 10.0 | 20.0 |
| Transparency | a | a | a |

As shown in Tables 9 and 10, in the aqueous ophthalmic compositions containing the castor oil and either the polyoxyethylene castor oil or the polyoxyethylene hydrogenated castor oil 60 (Comparative Examples 5 and 6), the transparency was low with transmittance of less than 75%. However, as shown in Tables 9 to 12, all of the aqueous ophthalmic compositions containing the castor oil and both the polyoxyethylene castor oil and the polyoxyethylene hydrogenated castor oil 60 (Examples 11-1 to 14-3) were transparent with transmittance of 75% or more.

As shown in Tables 9 to 12, it was confirmed that in the aqueous ophthalmic compositions containing the castor oil and both the polyoxyethylene castor oil and the polyoxyethylene hydrogenated castor oil 60, the higher the content ratio of the polyoxyethylene castor oil and the polyoxyethylene hydrogenated castor oil to the castor oil, the higher the transmittance.

### Preparation Examples

According to the formulations shown in Tables 13 and 14, eye drops (Preparation Examples 1 to 8), eye washes (Preparation Examples 9 and 10), and eye drops for soft contact lenses (Preparation Example 11) are prepared. Retinol palmitate is vitamin A palmitate manufactured by Wako Pure Chemical Industries, Ltd. (900,000 to 1,050,000 IU/g).

TABLE 13
Unit: w/v %

| | Preparation | Preparation | Preparation | Preparation | Preparation | Preparation |
|---|---|---|---|---|---|---|
| | Example 1 | Example 2 | Example 3 | Example 4 | Example 5 | Example 6 |
| | Eye drops | Eye drops | Eye drops | Eye drops | Eye drops | Eye drops |
| Polyoxyethylene | — | — | 0.01 | — | 0.5 | — |
| castor oil 3 | | | | | | |
| Polyoxyethylene | 0.1 | 0.5 | 0.1 | 0.5 | — | 0.25 |
| castor oil 10 | | | | | | |
| Castor oil | — | 0.1 | 0.5 | — | 0.05 | — |
| Retinol | 0.01 | — | 0.005 | 0.02 | 0.005 | 0.03 |
| palmitate | (10,000 | | (50,00 | (20,000 | (50,00 | (30,000 |
| | IU) | | IU) | IU) | IU) | IU) |
| Epsilon- | — | — | — | 3 | — | — |
| aminocaproic | | | | | | |
| acid | | | | | | |
| Berberine | 0.05 | 0.05 | — | 0.01 | 0.01 | — |
| sulfate hydrate | | | | | | |
| Chlorpheniramine | 0.03 | — | — | — | 0.03 | 0.015 |
| maleate | | | | | | |
| Pyridoxine | — | 0.1 | — | — | — | — |
| hydrochloride | | | | | | |
| Tocopherol | — | — | — | — | — | 0.01 |
| acetate | | | | | | |
| Potassium | 1 | 1 | — | 0.05 | 0.5 | — |
| L-aspartate | | | | | | |
| Aminoethylsulfonic | 1 | 0.05 | — | 0.1 | 0.5 | 1 |
| acid | | | | | | |
| Sodium | — | 0.2 | 0.1 | — | — | — |
| chondroitin | | | | | | |
| sulfate | | | | | | |
| Potassium | — | 0.15 | — | — | 0.04 | 0.08 |
| chloride | | | | | | |
| Calcium | — | — | — | — | — | 0.02 |
| chloride | | | | | | |
| Sodium chloride | 0.1 | 0.3 | — | — | 0.2 | 0.4 |
| Sodium hydrogen | — | — | 1.2 | — | — | — |
| phosphate | | | | | | |
| Sodium | — | — | 0.22 | — | — | — |
| dihydrogen | | | | | | |
| phosphate | | | | | | |
| Glucose | — | — | 0.2 | — | — | 0.1 |
| Boric acid | 0.4 | 1.2 | — | 0.6 | 1 | 0.5 |
| Borax | 0.1 | 0.3 | — | 0.1 | 0.25 | 0.1 |
| l-menthol | 0.03 | 0.004 | — | 0.008 | 0.005 | — |
| dl-camphor | — | — | 0.005 | 0.002 | 0.003 | — |
| d-borneol | — | — | 0.001 | — | 0.005 | — |
| Geraniol | 0.003 | — | — | — | — | — |
| Eucalyptus oil | — | — | — | 0.005 | — | — |
| Bergamot oil | 0.002 | 0.005 | — | — | — | — |
| Cool mint No. 71212 | — | 0.015 | — | — | 0.001 | — |
| Peppermint oil | — | — | — | — | — | 0.001 |
| Mentha oil | | — | 0.005 | — | — | — |
| Benzalkonium chloride | — | 0.01 | — | — | 0.005 | — |
| Polyhexanide | — | 0.0005 | — | — | — | — |
| hydrochloride | | | | | | |
| Methyl | — | — | — | 0.05 | — | — |
| parahydroxybenzoate | | | | | | |
| Ethyl | — | — | — | 0.026 | — | — |
| parahydroxybenzoate | | | | | | |
| Chlorobutanol | 0.05 | — | — | — | — | — |
| Polyoxyethylene | — | — | — | 0.25 | — | — |
| hydrogenated castor | | | | | | |
| oil 60 | | | | | | |
| Polysorbate 80 | 0.3 | — | 0.15 | — | 0.3 | 0.5 |
| Poloxamer 407 | 0.05 | — | — | 0.05 | 0.05 | — |
| Propylene glycol | 0.1 | — | 0.1 | 0.01 | — | — |
| Polyvinylpyrrolidone K25 | — | 0.2 | — | — | 1 | — |
| Hydroxyethyl | 0.05 | — | 0.1 | — | — | 0.2 |
| cellulose | | | | | | |
| Hypromellose | — | 0.1 | — | 0.05 | — | — |
| Sodium hyaluronate | 0.05 | — | 0.02 | — | — | — |
| Concentrated glycerin | 0.2 | — | 0.5 | 0.2 | — | — |
| Trometamol | — | 0.1 | 0.2 | — | — | 0.05 |
| Dibutylhydroxytoluene | — | 0.005 | — | — | — | — |
| (BHT) | | | | | | |
| Sodium edetate | 0.03 | — | — | — | 0.005 | — |
| Hydrochloric acid | q.s. | q.s. | q.s. | q.s. | q.s. | q.s. |
| Sodium hydroxide | q.s. | q.s. | q.s. | q.s. | q.s. | q.s. |
| Purified water | Balance | Balance | Balance | Balance | Balance | Balance |
| pH | 8.5 | 7 | 6.2 | 4.5 | 6 | 6.8 |

Note:
The figures in parentheses indicate the amount of vitamin A contained in 100 mL of each composition.

TABLE 14
Unit: w/v %

| | Preparation | Preparation | Preparation | Preparation | Preparation |
|---|---|---|---|---|---|
| | Example 7 | Example 8 | Example 9 | Example 10 | Example 11 |
| | Eye drops | Eye drops | Eye wash | Eye wash | Eye drops |
| | | | | | for SCL |
| Polyoxyethylene | 1 | 0.001 | 0.5 | 0.01 | 0.5 |
| castor oil 10 | | | | | |
| Castor oil | 0.025 | — | 0.001 | 0.05 | — |
| Retinol | — | 0.05 | — | — | 0.001 |
| palmitate | | (50000 IU) | | | (1000 IU) |
| Epsilon- | 2 | — | — | — | 0.5 |
| aminocaproic | | | | | |
| acid | | | | | |
| Berberine | 0.05 | — | — | 0.002 | — |
| sulfate hydrate | | | | | |
| Chlorpheniramine | — | — | 0.003 | — | — |
| maleate | | | | | |
| Pyridoxine | 0.05 | — | 0.01 | — | — |
| hydrochloride | | | | | |
| Tocopherol | — | 0.05 | 0.005 | — | 0.01 |
| acetate | | | | | |
| Potassium | 1 | — | 0.1 | — | 1 |
| L-aspartate | | | | | |
| Sodium | 0.5 | — | 0.025 | 0.05 | — |
| chondroitin | | | | | |
| sulfate | | | | | |
| Potassium | 0.01 | — | 0.08 | — | 0.08 |
| chloride | | | | | |
| Calcium | 0.005 | — | — | — | 0.015 |
| chloride | | | | | |
| Sodium chloride | — | 0.5 | 0.4 | 0.1 | 0.4 |
| Boric acid | 0.5 | — | 1.6 | 0.5 | 0.1 |
| Borax | 0.1 | — | 0.35 | 0.01 | 0.1 |
| l-menthol | 0.004 | 0.002 | 0.0005 | 0.001 | 0.02 |
| dl-camphor | — | 0.002 | — | — | 0.0001 |
| dl-borneol | — | 0.005 | — | — | — |
| Geraniol | — | — | 0.005 | 0.001 | — |
| Eucalyptus oil | — | — | — | 0.001 | 0.001 |
| Bergamot oil | — | — | — | — | 0.002 |
| Peppermint oil | — | — | 0.02 | — | — |
| Mentha oil | — | 0.01 | — | — | — |
| Potassium sorbate | 0.05 | — | 0.1 | — | — |
| Polyhexanide | 0.0005 | — | — | — | 0.001 |
| hydrochloride | | | | | |
| Chlorobutanol | — | — | — | 0.2 | 0.4 |
| Polyoxyethylene | 0.2 | — | — | 0.1 | — |
| hydrogenated castor | | | | | |
| oil 60 | | | | | |
| Polysorbate 80 | — | 0.1 | 0.1 | 0.3 | — |
| Poloxamer 407 | 0.1 | — | 0.05 | — | 0.05 |
| Hydroxyethyl | — | — | — | — | 0.1 |
| cellulose | | | | | |
| Hypromellose | 0.2 | — | — | — | — |
| Sodium hyaluronate | — | — | 0.02 | — | — |
| Concentrated glycerin | — | — | — | — | 0.2 |
| Trometamol | — | — | — | 1 | — |
| Dibutylhydroxytoluene | 0.005 | — | 0.005 | — | — |
| (BHT) | | | | | |
| Sodium citrate | — | 0.5 | 0.5 | — | — |
| Sodium edetate | 0.05 | 0.02 | — | 0.03 | 0.01 |
| Hydrochloric acid | q.s. | q.s. | q.s. | q.s. | q.s. |
| Sodium hydroxide | q.s. | q.s. | q.s. | q.s. | q.s. |
| Purified water | Balance | Balance | Balance | Balance | Balance |
| pH | 5.5 | 7 | 7.5 | 6.5 | 6.5 |

Note:
The figures in parentheses indicate the amount of vitamin A contained in 100 mL of each composition.

1. A method for reducing defoaming time in an aqueous ophthalmic composition, comprising adding (A) polyoxyethylene castor oil in which the number of moles of added ethylene oxide is 2 to 30, and (B) at least one member selected from the group consisting of castor oil and vitamin A to the aqueous ophthalmic composition.

2. The method according to claim 1, further comprising adding (C) at least one member selected from the group consisting of boric acids and salts thereof to the aqueous ophthalmic composition.

3. The method according to claim 1, further comprising adding (D) at least one member selected from the group consisting of glycerin and nonionic surfactants other than component (A) to the aqueous ophthalmic composition.

4. The method according to claim 3, wherein each components are added to the aqueous ophthalmic composition such that the total content of component (A) and component (D) is 2 to 50,000 parts by weight relative to 1 part by weight of the total content of component (B).

5. A method for reducing variation in drip amount during use of an aqueous ophthalmic composition, comprising adding (A) polyoxyethylene castor oil in which the number of moles of added ethylene oxide is 2 to 30, and (B) at least one member selected from the group consisting of castor oil and vitamin A to the aqueous ophthalmic composition.

6. The method according to claim 5, further comprising adding (C) at least one member selected from the group consisting of boric acids and salts thereof to the aqueous ophthalmic composition.

7. The method according to claim 5, further comprising adding (D) at least one member selected from the group consisting of glycerin and nonionic surfactants other than component (A) to the aqueous ophthalmic composition.

8. The method according to claim 7, wherein each components are added to the aqueous ophthalmic composition such that the total content of component (A) and component (D) is 2 to 50,000 parts by weight relative to 1 part by weight of the total content of component (B).

9. An aqueous ophthalmic composition, comprising (A) polyoxyethylene castor oil in which the number of moles of added ethylene oxide is 2 to 30, and (B) at least one member selected from the group consisting of castor oil and vitamin A.
